(19) United States
(12) Patent Application Publication
(10) Pub. No.: US 2023/0064184 A1
(43) Pub. Date: Mar. 2, 2023

(54) FOAM COMPOSITIONS FOR THE TREATMENT OF CANCER

(71) Applicant: TDL Innovations, LLC, Princeton, NJ (US)

(72) Inventor: Glenn W. Laub, Princeton, NJ (US)

(21) Appl. No.: 17/760,073

(22) PCT Filed: Feb. 3, 2021
(86) PCT No.: PCT/US21/16487

§ 371 Date: Aug. 3, 2022

Related U.S. Application Data
(60) Provisional application No. 62/969,493, filed on Feb. 3, 2020.

(51) Int. Cl. A61K 9/12; A61K 39/07; A61K 31/704; A61K 31/337; A61K 31/407; A61K 31/7068; A61K 31/395; A61K 31/282; A61K 33/243; A61K 31/519; A61P 35/00; A61M 11/02

(52) U.S. Cl. CPC A61K 9/122; A61K 39/07; A61K 31/704; A61K 31/337; A61K 31/407; A61K 31/7068; A61K 31/5545; A61K 31/282; A61K 33/243; A61K 31/519; A61P 35/00; A61M 11/02; A61K 2039/585

(57) ABSTRACT

Disclosed is the use of foam compositions for the delivery of therapeutics to a body cavity, organ, or tissue of a patient in need thereof. Also disclosed are foam compositions, and devices for delivering the composition to a body cavity or the interior or exterior of an organ. Additionally, methods of treating cancer in the internal organs or the body cavity using the foam compositions are also disclosed.

### CROSS-REFERENCE TO RELATED APPLICATIONS

This application claims the benefit of U.S. Provisional Application No. 62/969,493, filed Feb. 3, 2020, which is incorporated herein by reference.

### FIELD

The present disclosure provides use of foam compositions for the delivery of anti-cancer therapeutics to a body cavity, organ, or tissue of a patient in need thereof Also disclosed are foam compositions, and devices for delivering the composition to a body cavity or the interior or exterior surface of an organ or tissue. Additionally, methods of treating cancer involving an internal organ or in a body cavity using the foam compositions are disclosed.

### BACKGROUND

Cancer is a generic term for a group of diseases featuring rapid creation of abnormal cells that grow beyond their usual boundaries, and which can then invade adjoining parts of the body and spread to other organs. Carcinoma, i.e., the cancer of the skin, body cavity (e.g., peritoneal cancer), and internal organs (e.g., bladder cancer, lung cancer, mesothelioma) is the most prevalent cause of cancer-related deaths.

Currently, the primary treatment approach of treating cancers present in an internal organ such as bladder and lung, or in a body cavity, such as the peritoneal cavity, is systemic administration of therapeutic agents (e.g., by oral or intravenous routes). Systemic administration, however, has some disadvantages. Oral administration, for example, suffers from a first-pass effect, i.e., the concentration of the therapeutic agent may be reduced before reaching the affected area due to metabolism in the liver. Consequently, the process becomes inefficient, requiring a high dose and causing side effects.

Even when cancer therapeutic agents are administered directly to the affected cavity or internal organ, the process has significant limitations. Direct administration typically involves either aerosol therapy or the use of a catheter to deliver a liquid containing the therapeutic agent to the affected area. Both of these methods have certain disadvantages. Aerosol therapy can only be used in limited areas of the body, such as lung or pleural space. Additionally, during aerosol therapy, very low dose of therapeutic agents can target airflow, which may not cover the target area completely. Moreover, if the patient has constricted airway due to a disease such as asthma, aerosol cannot be inhaled effectively. Delivery of the therapeutic agent in a liquid form via a catheter also has significant limitations. After delivery of the liquid containing the therapeutic agent to the targeted area, the liquid tends to accumulate at certain locations due to gravity causing uneven distribution of the therapeutic agent in the affected area. In addition, the liquid may gather in a tissue or location where treatment is not required causing ineffective treatment and unintended side effects.

In view of the current treatment options, there is an unmet need for a delivery mechanism where the therapeutic agent, in particular an anti-cancer agent, is effectively distributed to the target location, organ or tissue, allowing higher local tissue concentration than systemic administration and causing minimal side effects. The present disclosure provides foam compositions for administration of one or more anti-cancer agents to an organ, tissue, or body cavity, which can allow effective distribution of the anti-cancer agent or agents in the affected area while minimizing side effects.

### SUMMARY

In one aspect, the disclosure provides a method to treat a neoplastic disease or condition in a patient in need thereof by administering a foam composition comprising a foaming agent and further comprising one or more therapeutic agents, in particular anti-cancer agents. The neoplastic disease or condition may be cancer, including cancer of an internal organ or a body cavity. In some embodiments, the cancer is bladder cancer, lung cancer, mesothelioma, or peritoneal cancer. The cancer may be contained in the location of its origin or it may have metastasized to other locations of the body. In some embodiments, the cancer involves the bladder, lung, pleural space, or peritoneal cavity and may have metastasized from other locations of the body. Additionally, cancer may feature carcinomatosis (as in, for example, peritoneal carcinomatosis). In some embodiments, the cancer may be localized, in others it be a stage 3 or stage 4 cancer.

In one embodiment, the disclosure provides a method to treat bladder cancer in a patient in need thereof comprising administering a foam composition comprising a foaming agent, and further comprising Bacillus Calmette-Guérin (BCG) and/or valrubicin to the bladder.

In another embodiment, the disclosure provides a method to treat lung cancer comprising administering a foam composition comprising a foaming agent and further comprising one or more of cisplatin, oxaliplatin, doxyrubicin, paclitaxel, and mitomycin C to the lung. The cancer may be an advanced stage cancer. In other embodiments, the cancer is localized.

In another embodiment, the disclosure provides a method to treat peritoneal cancer comprising administering a foam composition comprising a foaming agent and further comprising one or more of mitomycin C, paclitaxel, taxotere, oxaliplatin, and cisplatin to the peritoneal cavity.

In another embodiment, the disclosure provides a method to treat pleural cancer comprising administering a foam composition comprising a foaming agent and further comprising one or more of pemetrexed (Alimta®), cisplatin, carboplatin, gemcitabine (Gemzar®), vinorelbine, doxorubicin, oxaliplatin, and paclitaxel to the pleural cavity.

In some aspects, the foam composition is sterile, i.e., free, or substantially free of, living microbes and/or bacteria. All ingredients of the composition may be sterilized separately, mixed, and packaged under aseptic conditions. Alternatively, the composition may be sterilized in its final form prior to administration. In other embodiments, the compositions comprise a pharmaceutically acceptable carrier and a cellulose derivative, which is heat sterilizable.

The foam compositions disclosed herein are capable of forming a foam before, during, or after administration of the foaming composition to the target organ, tissue or body cavity. In addition to an active agent, e.g., an anti-cancer agent, the foam composition comprises a liquid component, one or more foaming agents (e.g., a surfactant or polymer) and one or more bubble-forming agents (e.g., a dissolved gas, or volatile liquid that forms a gas upon exposure to ambient temperature and/or body temperature and ambient pressure). The gas component may be a single gas or a mixture of gasses. The volatile liquid may be a fluorinated hydrocarbon (for example, H134a or HFO1234ze).

The foam (or foamable) compositions disclosed herein comprises one or more anti-cancer therapeutic agents including chemotherapeutic agents, anti-inflammatory agents, antibodies, antimetabolites, hormones, vaccine, immunostimulants. The composition may further comprise, anti-infective agents including antibiotics, imaging agents, hemostatic agents, gene therapy including vectors, hemolytic agents, adhesion reducing agents, adhesives, anti-inflammatory agents, and/or joint lubricants.

In some embodiments, the anti-cancer agent is a chemotherapeutic agent. Chemotherapeutic agents may include one or more of the following agents, calrubicin, doxorubicin, paclitaxel, mitomycin C, gemcitabine (Gemzar®), vinorelbine, taxotere, carboplatin, cisplatin, oxaliplatin, pemetrexed (Alimta®). In other embodiments, the anti-cancer agent is an immunostimulant, for example Bacillus Calmette-Guérin (BCG).

In another aspect, the disclosure provides a device for delivery of a foam composition disclosed herein comprising one or more reservoirs for the therapeutic agent and propellant, a foam generator, regulator, actuator, and patient interface (such as a catheter or tube, e.g., as described herein). The device may comprise one or more containers, e.g., two containers. The reservoir for the therapeutic agent may be one of the containers. The device may be a single use device or may be used multiple times.

In some embodiments, the device comprises two containers comprising (i) a foamable composition with one or more therapeutic agents and (ii) a pressurized gas canister. In other embodiments, the device comprises containers comprising (i) a foamable composition and (ii) one or more therapeutic agents and pressurized gas. In such embodiments comprising two containers, the compositions in the separate containers are combined prior to administering to a tissue, organ, or body cavity. Alternatively, the devices comprise a single container with (a) the liquid component, foaming agent, one or more therapeutic agents and propellant, or (b) the liquid component, foaming agent, one or more therapeutic agents, and dissolved gas. In another aspect, the therapeutic agent and/or propellant can be added to the device through a valve or port prior to administration.

### BRIEF DESCRIPTION OF THE DRAWINGS

FIG. 1 shows results from 30 second images of luciferase activity on Day 7 (top panel) and Day 10 (bottom panel). These results suggest that the foam with the chemotherapeutic agent (mitomycin C) provides a significant reduction in tumor activity compared with MMC or HIPEC with MMC on Day 7. On Day 10 the foam MMC composition provided a significant reduction in tumor activity compared with HIPEC with MMC and a reduction in tumor activity compared to MMC that did reach statistical significance at this sample size.

FIG. 2 shows a survival plot on the control, sham and treatment groups. The survival of sham and treatment groups is better than the control group.

FIG. 3 shows representative images of Control mice (top left), Sham L23 mice (top right), MMC treated mice (2^{nd} row), MMC L23 treated mice (3^{rd} row), and MMC HIPEC treated mice (bottom row). Prior to imaging, mice were injected IP with 100 ml of luciferin substrate 30 mg/ml solution and Luciferase activity was imaged 5 min after substrate injection. Luciferase activity was analyzed on IVIS Lumina-III pre-clinical in vivo imaging system.

### DETAILED DESCRIPTION

The present disclosure provides a composition for the delivery of one or more anti-cancer therapeutics to a body cavity, organ, or tissue of a patient in need thereof Also disclosed are compositions and devices for delivering the composition to a body cavity or to the interior or exterior of an organ. Additionally, methods of treating diseases using the foam compositions described herein are disclosed.

Delivering a therapeutic agent directly to the body cavity, internal organ, or tissue of a patient allows higher local tissue concentration of the anti-cancer agent than systemic administration (e.g., by oral or i.v. route), which allows the therapeutic agent to be more effective while limiting the side effects. The foam compositions described herein effectively deliver the therapeutic agent directly to the organ or tissue in need of treatment. Systemic drug administration often requires high dosages or repeated administration to achieve a therapeutic effect, which increase the risk of side effects and lower patient tolerance. The present disclosure provides foam-based delivery systems containing an anti-cancer agent that is delivered directly to the tissue or organ thereby providing increased local tissue concentrations of the anti-cancer agent than would be achieved by systemic administration. Additionally, administering the therapeutics agents in a foam composition can provide spatial and temporal control over the delivery and release of the therapeutic agents.

Foam Compositions

The foam compositions contain at least one active ingredient in a biocompatible foamable carrier including but not limited to oleaginous foams, oil-in-water foams, water-in-oil foams, liposome-based foams, surfactant-based foams, and nanoparticle-based foams. As used herein a foam composition includes a foamable composition, or a composition capable of forming a foam composition. In some embodiments, the foam comprises a liquid component, a foaming agent, and a bubble-forming agent, which are configured to form a foam. The foam composition further comprises a therapeutically effective amount of one or more active therapeutic agents (in particular an anti-cancer agent) that may be dissolved, dispersed, or suspended in the liquid component. The foam compositions may further comprise one or more solvents, stabilizers, and preservatives. In some embodiments, the composition may optionally comprise a foam adjuvant.

In one aspect of the disclosure, the composition is sterile (e.g., substantially free of microbes and/or bacteria). In embodiments, ingredients of the composition are sterilized separately, mixed, and packaged under aseptic conditions. In other embodiments, the compositions comprise a pharmaceutically acceptable carrier which is heat sterilizable. In some embodiments, the composition is at least 80%, at least 90%, and up to 100% sterile.

In some embodiments, the foam formed by the foam composition is stable. In some embodiments, the foam retains at least 50%, at least 70%, at least 85%, at least 90%, at least 95%, or at least 99% of the original foam volume after two to four minutes of administration; after four to ten minutes of administration; or after ten to fifteen minutes of administration of the composition and foam formation. In some cases the foam persists for 1 to 2 hours, 2 to 10 hours, 10 to 24 hours, 1 to 2 days, 2 to 14 days, or several weeks after the administration of the composition and foam formation.

The foam compositions include a liquid component, such as one or more of water, a non-aqueous solvent, and/or a hydrophobic organic carrier. The hydrophobic organic carrier may be propylene glycol. The compositions may comprise a solvent and/or an emulsifier, including but not limited to methylparaben, propylparaben, and emulsifying wax. The compositions may optionally comprise a stabilizer, viscosity modifier, or bio-adhesive agent such as xanthan gum, locust bean, gum arabic, gum ghatti, guar gum, gum tragacanth, karaya gum, pullulan, alginate, carrageenan, pectin, gellan, chitosan, chondroitin sulfate, dermatin sulfate, and heparin.

The foam compositions also include one or more foaming agents that are present in the foam composition to provide structure and allow bubbles to be trapped in the composition so that when a gas is evolved, a foam results. Thus, the foaming agent may be, for example, a polymer or a surfactant. The foam composition may contain one, two, three or more foaming agents. The foaming agent may be a surfactant including a biocompatible non-ionic surfactant, e.g., polysorbate 80, Laureth 23 (Lipocol L-23), or Poloxamer. In some embodiments, the foaming agent may be a natural surfactant (e.g., albumin, gelatin, denatured collagen, soybean-derived proteins, or stearic acid).

In some embodiments, the composition may comprise one or more biocompatible surfactants. The one or more surfactants may include a biocompatible foaming agent selected to modify the stability of the foam formed after administering the composition to a patient. In some embodiments, the surfactants may further function as a sclerosing agent. Examples of such surfactants include phospholipids, neutral lipids, hydrophobic surfactants, biocompatible soaps or detergents, and combinations thereof. In some embodiments, the surfactant may be sodium tetradecyl sulfate, polysorbate 80, or poloxamer.

Poloxamers comprise co-polymers of a hydrophobic poloxypropylene (POP) moiety sandwiched between two hydrophilic moieties of poloxyethylene (POE) and have the ability to solubilize lipophilic drugs within the hydrophobic core. Poloxamer based gel/foam formulations exhibit thermosensitive rheological properties, which may be advantageous for localized, sustained delivery of drugs. The foam composition may comprise one or more Poloxamer polymers selected from the group consisting of Poloxamer 407 (F127), Poloxamer 338 (F108), and/or Poloxamer 188 (F68).

In some embodiments, the foam composition can be in the form of a thermo-sensitive gel. In some embodiments, the viscosity of the composition increases above a specific temperature leading to a decreased diffusion of any therapeutic agents present in the composition leading to sustained delivery and prolonged localized “depot” in the targeted area of treatment. In embodiments where the foaming agent is a thermosensitive polymer or gelling agent, the viscosity of composition increases during or after administering the composition to a tissue, organ or body cavity. In some embodiments, the foam composition comprises a gel or a composition which forms a gel, that is substantially liquid (low viscosity) at about room temperature (e.g., about 20° C. to about 25° C.), but as the temperature of the composition increases, the viscosity of the composition increases. In some embodiments, the composition has a viscosity of less than 800,000 centipoise (cP), less than 750,000 cP, less than 200,000 cP, less than 150,000 cP, less than 100,000 cP, less than 90,000 cP, less than 70,000 cP, 50,000 cP, less than 30,000 cP, less than 10,000 cP, less than 1000 cP, less than 500 cP, less than 100 cP, less than 50 cP, less than 10 cP, less than 5 cP, less 1 cP, less than 0.1 cP. In embodiments, the viscosity of the composition increases between 25° C. and 40° C. In some embodiments, the increased viscosity at a higher temperature is about 10,000 cP, about 20,000 cP, about 50,000 cP, about 100,000 cP, about 200,000 cP, about 500,000 cP, about 750,000 cP, about 1,000,000 cP.

In embodiments, the foam composition is a dissolved gas foam in which the bubble-forming component is a gas or mixture of gasses that is dissolved in the liquid component when under pressure, but form gas bubbles when exposed to ambient pressure. In some embodiments, the dissolved gas foam may comprise a single gas such as nitric oxide, nitrous oxide, carbon dioxide or oxygen, or a mixture of gases. The gas present in the foam is preferably uniformly dispersed. The gases in the composition may be present as microbubbles, i.e., with small microscopic bubbles not visible to the naked eye. As used herein, the term foam encompasses foams with bubbles of all sizes, including micro foams.

In embodiments, the composition comprises a liquid component (e.g., comprising water) and one or more components that form a non-cross linked polymer network, for example, during or after administration to a patient. In some embodiments, the composition has between 50-99% water content providing physical similarity to the targeted tissues. In some embodiments, the composition may be a gel or a composition, which forms a gel (e.g., a hydrogel). In some embodiments, the foam is biodegradable. In some embodiments, the non-cross linked foams have the properties of cross-linked foam products such as non-abrasion and soft touch.

In some embodiments, the composition comprises a liquid component (e.g., comprising water) and one or more components that form a cross-linkable polymer network, for example, during or after administration to a patient. The cross-linking of polymers may occur through chemical means such as initiation of polymerization either by contact as in cyanoacrylates or by external stimuli such as photo-initiation. Gel formation can also be achieved with low molecular weight cross-linkers such as glutaraldehyde or carbodiimide. In another embodiment, the cross-linking may be achieved by using a physical approach such as self-assembling peptides. The composition may comprise one or more biological components such as mussel glue, fibrin tissue adhesives, factor XII, calcium-independent microbial transglutaminase (mTG), and/or gelatin for cross-linking the polymers.

In some aspects, the foam composition comprises a bubble-forming component that comprises a volatile liquid that remains a liquid under pressure, but forms a gas under ambient pressure and/or ambient temperature or body temperature. The composition may comprise one or more volatile liquids that provide gas at the gas-forming temperature/pressure. These include, for example, fluorinated organic compounds, such as fluorinated, including perfluorinated, hydrocarbons, hydrofluorocarbons and hydrofluoroolefins. Examples of such fluorinated organic compounds include trifluoromethane, difluoromethane, difluoroethane, tetrafluoroethane, heptafluoroethane, perfluorobutane, perfluorocyclobutane, perfluoropentane, perfluorohexane, perfluoroheptane, perfluorooctane, perfluorocyclopentane, perfluorocyclohexane, heaxafluoropropane, and/or heptafluoropropane. In some embodiments, the volatile liquid may be a propellant such as a haloalkane, including 1,1,1,2-tetrafluoroethane (H134A) and/or a hydrofluoroolefin such as 1,3,3,3-tetrafluoropropene (HFO-1234ze(E)). Other volatile liquids that can be used as the bubble forming component of the foam compositions include sulfur hexafluoride; alkanes, such as propane, butanes and pentanes; cycloalkanes and cycloalkenes, such as cyclobutane, cyclopentene, and cyclohexene; dialkyl ethers, such as dimethyl ether, methyl ethyl ether, and diethyl ether; cycloalkylene ethers, such as furan; ketones, such as acetone and methyl ethyl ketone; and carboxylates, such as formic acid, acetic acid, and propionic acid. All such agents may be used in these aspects of the present disclosure.

In some aspects, the composition may comprise a chemical foam where the bubble forming component comprises one or more chemicals that react to form a gas.

In embodiments, the foam composition comprises one or more therapeutic agents, 10-30% poloxamer in water, 0.1-3% xanthan gum, and/or 1-25% PEG. In some embodiments, the foam composition comprises one or more therapeutic agents, 70-90% purified water, 2-10% polysorbate 80, 0.01-1.5% methylparaben, 5-10% propylene glycol and 0.05-5.0% xanthan gum, and/or 2.5-50% H134A. In some other embodiments, the foam composition comprises one or more therapeutic agents, 60-90% purified water, 5-15% propylene glycol, 0.1-1% xanthan gum, 0.2-1.5% Lipocol L-23, 0.2-2% emulsifying wax, 0.1-1% cetyl alcohol, 0.01-0.1% methylparaben, 0.01-0.05% propylparaben, and/or 5-50% H134A.

In embodiments, the foam composition comprises one or more therapeutic agents, about 10% to about 30% poloxamer in water, about 0.1% to about 3% xanthan gum, and/or about 1% to about 25% PEG. In some embodiments, the foam composition comprises one or more therapeutic agents, about 70% to about 90% purified water, about 2% to about 10% polysorbate 80, about 0.01% to about 1.5% methylparaben, about 5% to about 10% propylene glycol, about 0.05% to about 5.0% xanthan gum. In some embodiments, the foam composition comprises one or more therapeutic agents, about 5% to about 20% propylene glycol, about 0.1% to about 1% xanthan gum, about 0.2% to about 1.5% Lipocol L-23, about 0.2% to about 2% emulsifying wax, about 0.1% to about 1% cetyl alcohol, about 0.01% to about 0.1% methylparaben, and/or about 0.01% to about 0.05% propylparaben. In embodiments, the foam composition comprises one or more therapeutic agents about 15% propylene glycol, about 0.5% Lipocol L-23, about 0.5% emulsifying wax, about 0.3% cetyl alcohol, about 0.05% methylparaben, about 0.01% propylparaben, and about 0.3% xanthan gum. Percentages of components in the compositions provided herein are in percent weight by weight of the composition (% w/w) unless specified otherwise.

Therapeutic Agents

In one aspect, the composition comprises one or more anti-cancer therapeutic agents including chemotherapeutic agents, anti-inflammatory agents, antibodies, antimetabolites, hormones, vaccine, immunostimulants, and combinations thereof. The composition may further comprise, anti-infective agents including antibiotics, imaging agents, hemostatic agents, gene therapy agents including vectors, hemolytic agents, adhesion reducing agents, adhesives, anti-inflammatory agents, and/or joint lubricants. In some embodiments, the chemotherapeutic agent is selected from the group consisting of Bacillus Calmette-Guérin (BCG), valrubicin, doxorubicin, paclitaxel, mitomycin C, gemcitabine (Gemzar®), vinorelbine, taxotere, carboplatin, cisplatin, oxaliplatin, and pemetrexed (Alimta®).

Patient Population

The patient (or subject) may be a human or other non-human mammal. The human patients are typically suffering from a neoplastic disease or condition where the neoplastic disease or condition is cancer. In some embodiments, the patient is suffering from bladder cancer, lung cancer, pleural cancer, mesothelioma, or peritoneal cancer and/or cancer that may have metastasized from other locations of the body. In some embodiments, the cancer is localized, in others it is an advanced stage cancer, i.e., stage 3 or stage 4 cancer. The cancer may additionally feature carcinomatosis.

Dosage

An effective amount or a therapeutically effective amount of the composition is an amount sufficient to confer a therapeutic benefit in a patient after administration, for example, to improve in the subject one or more symptoms of the disease. The effective amount may vary depending on the species, age, weight, and/or health of the subject and the nature or severity of the disease. In some cases, multiple doses of the composition are administered to achieve the effective amount for the therapeutic benefit intended. In some embodiments, the amount of therapeutic agent present in a single dose of the foaming compositions described herein is about 10%, about 20%, about 30%, about 40%, about 50%, about 60%, about 70%, about 80%, or about 90% less than the amount of therapeutic agent present in a single dose used for systemic administration for treating a similar disease at similar stage.

Devices for Delivering Foams Generally

The devices for delivery of the foam compositions comprise one or more components selected from a reservoir for the therapeutic agent and/or propellant, a foam generator, regulator, actuator, and patient interface.

In one aspect of the disclosure, the device used for the delivery of the composition comprises one or more containers, e.g., two containers. In some embodiments, the device may comprise containers comprising (i) a foamable composition with one or more therapeutic agents and (ii) a pressurized gas canister; or (i) a foamable composition and (ii) one or more therapeutic agents and pressurized gas.

In some embodiments, the contents of the two containers are combined prior to delivery to the patient or during delivery to the patient. In some specific embodiments, one container is used to charge, or pressurize, the other container before administering the final composition in a tissue, organ or body cavity. In some embodiments, the compositions of the containers are combined before being administered into the patient's body. In some embodiments, the compositions of the containers are administered into the patient's body in tandem or subsequent to each other.

In some aspect, the disclosure comprises a composition in a single container comprising one or more therapeutic agents, a liquid component, a foaming agent and bubble forming component such as a dissolved gas or volatile liquid. In embodiments, the therapeutic agent can be added to the device through a valve or port prior to administration. In addition, or alternatively, the gas or the propellant can be added to the device through a valve or port prior to administration.

In some aspects of the present disclosure, the device used for the delivery of the composition is a single-use device. In other aspects, the device used for the delivery of the composition can be used multiple times.

In some embodiments, the container outlet is attached to a catheter such that the composition is delivered from the container to the patient through the catheter. A tube, such as a catheter, may be a thin pliable tube that can be inserted into the body. In embodiments, the composition may be introduced to a patient's body using an injector, ejector, aspirator pump, eductor-jet pump or other device. In embodiments, such devices employ a Venturi effect (the reduction in fluid pressure that results when a fluid flows through a constricted section of a tube).

Methods of Treatment

The subjects receiving the therapy described herein (e.g., a therapeutically effective amount of a foam composition, or compositions, comprising one or more anti-cancer agents) may experience because of the therapy, a reduction or absence of the neoplastic disease or condition. In one aspect, the disclosure provides a method of treating, preventing and/or reducing the severity of a neoplastic disease or condition by administering a therapeutically effective amount of foam composition comprising an anti-cancer agent into a patient's body in need thereof. As used herein, “treating,” “treat,” and “treatment” refer to reducing, relieving, ameliorating, or alleviating at least one of the symptoms of the disease or disorder. For example, in respect to treating cancer, “treating,” “treat,” “treatment” refers to, for example, reducing tumor size, slowing progression of cancer, and/or reducing or inhibiting metastasis.

The terms “prevent,” “prevention” and the like refer to acting prior to overt disease or disorder onset, to prevent the disease or disorder from developing, or to minimize the extent of the disease or disorder, or slow its course of development. For example, in respect to treating cancer, “prevent,” “prevention” and the like may refer to stopping a tumor from recurring or inhibiting metastasis. The term “cure,” “curing” and the like refer to heal, to make well, or to restore to good health or to allow a time without recurrence of disease so that the risk of recurrence is small.

The phrase “therapeutically effective amount” is used herein to mean an amount sufficient to results in a desired beneficial change of physiology in the subject or to cause an improvement in a clinically significant condition in the subject, for example, by delaying, reducing, minimizing, or mitigating one or more symptoms associated with the disease or disorder.

The foam compositions described herein are administered to treat a neoplastic disease or condition, such as cancer. Cancer is a group of diseases in which abnormal cells divide without control. Many cancers can invade nearby tissues. Cancer cells can also spread to other parts of the body through the blood and lymph systems via metastasis. Carcinoma is a type of cancer that begins in the skin or in tissues that line or cover internal organs or in the body cavities. In a specific aspect, the disclosure provides a method for treating carcinoma by administering a foam composition comprising an anti-cancer agent to a tissue, organ, or body cavity of a patient in need thereof. In embodiments, the disclosure provides a method for treating cancer present in the internal organs or body cavity of a patient by administering a foam composition comprising an anti-cancer agent to the affected tissue or organ of the patient in need thereof.

In some embodiments, the composition is delivered to the internal or external surfaces of an internal organ including but not limited to mesothelium, bladder, ovary, and/or lung. In some embodiments, the composition is delivered to the body cavity of a patient in need thereof including, but not limited to, delivery to the peritoneal cavity, pleural space, abdominal cavity, and/or abscess cavity.

Methods of Treating Bladder Cancer

In certain aspects, the disclosure provides methods for treating bladder cancer by administering a foam composition described herein to the bladder of a patient in need thereof. In embodiments, the foam composition comprises one or more of BCG and valrubicin.

The bladder cancer treated using the present disclosure may be urothelial cancer, squamous cell cancer, adenocarcinoma, sarcoma, or small cell bladder cancer. The bladder cancer may be non-invasive, such as invasive papillary carcinoma and carcinoma in situ (CIS), non-muscle-invasive, such as neoplastic growth only on the lamina propria, or muscle-invasive, such as neoplastic growth on the bladder's wall muscle and/or into the fatty layers or surrounding tissue outside the bladder. The non-muscle-invasive bladder cancer may spread into the bladder muscle or to other parts of the body. Additionally, all cell types of bladder cancer can spread beyond the bladder to other areas of the body through metastasis. In some embodiments, the bladder cancer may spread from the sub-peritoneal space superior and posterolateral to the peritoneal cavity via the peritoneal lining. In other embodiments, the bladder cancer is localized.

The foam compositions described herein can be administered to the bladder using the devices described herein that have been adapted for delivery of the foam composition to the bladder. In some embodiments, the composition may be introduced directly to the target area of treatment in a patient's body using the device. In some embodiments, the container outlet of the device may be attached to a catheter such that the composition is delivered from the container to the patient through the catheter.

A tube, such as a catheter may be a thin pliable tube that can be inserted into the body. In some embodiments, the catheter may be a soft hollow tube, which is passed into the bladder to drain or introduce an agent in the bladder. The catheter used in the present disclosure may be an indwelling catheter including a urethral indwelling catheter and a suprapubic indwelling catheter or an intermittent catheter including a non-hydrophilic or a hydrophilic catheter. The catheter may be a standard bladder catheter. The foam composition can be administered to the bladder once or multiple times.

Methods for Treating Lung Cancer

In certain aspects, this disclosure provides methods of treating lung cancer comprising administering a foam composition described herein to a lung of a patient in need thereof. In embodiments, the foam composition comprises one or more of cisplatin, oxaliplatin, doxyrubicin, paclitaxel, and mitomycin C.

The lung cancer treated using the present disclosure may be small cell lung cancer or non-small cell lung cancer including adenocarcinoma, squamous cell carcinoma, large cell carcinoma, and undifferentiated non-small cell lung cancer. Additionally, all cell types of lung cancer can spread beyond the lung to other areas of the body through metastasis. In some embodiments, the lung cancer may spread to the outer surfaces of the lungs and the chest wall. In some aspects, the lung cancer treated using the present disclosure is either stage 3 or stage 4 of lung cancer. In other aspects, the lung cancer treated using the present disclosure is localized.

The foam compositions described herein can be administered to the lung using the devices described herein that have been adapted for delivery of the foam composition to the lung. In embodiments, the device may comprise a pressurized canister or an aerosol container. The foam can be administered directly to the area during surgery or subsequently through a catheter placed either during or after surgery. The catheter can be a chest tube, indwelling pleural catheter or other type of catheter. The foam composition can be administered to the patient's lung(s) once or multiple times.

Methods for Treating Mesothelioma

In certain aspects, this disclosure provides methods of treating mesothelioma by administering a foam composition to the pleural space of a patient in need thereof. In embodiments, the foam composition comprises one or more of the pemetrexed (Alimta®), cisplatin, oxaliplatin, carboplatin, gemcitabine (Gemzar®), vinorelbine, doxorubicin, paclitaxel, or combinations thereof.

Mesothelioma is a type of cancer that occurs in the mesothelium, i.e., the thin layer of tissue that covers the majority of the internal organs. The mesothelioma treated using the foam compositions of the present disclosure may include pleural mesothelioma, peritoneal mesothelioma, cardiac mesothelioma or testicular mesothelioma. Additionally, all cell types of mesothelioma cancer can spread beyond the primary neoplastic growth site to other areas of the body through metastasis. In some embodiments, the mesothelioma treated using the present disclosure is pleural mesothelioma. In some embodiments, the mesothelioma is caused by inhalation of asbestos.

The foam compositions described herein can be administered to the mesothelium using the devices described herein that have been adapted for delivery of the foam composition to the mesothelium. The foam can be administered directly to the area during surgery or subsequently through a catheter placed either during or after surgery. The catheter can be a chest tube, indwelling pleural catheter or other type of catheter. The foam composition can be administered to the patient's the mesothelium once or multiple times.

Methods for Treating Peritoneal Cancer

In certain aspects, this disclosure provides methods of treating peritoneal cancer by administering a foam composition to the peritoneal space of a patient in need thereof. In embodiment, the foam composition comprises one or more of the following agents, BCG, valrubicin, doxorubicin, paclitaxel, oxaliplatin, mitomycin C, gemcitabine (Gemzar®), vinorelbine, taxotere, carboplatin, cisplatin, pemetrexed (Alimta®) and/or any other not yet identified agents.

Peritoneal cancer refers to a cancer that has spread to the peritoneal lining of the peritoneal cavity. The cancer may spread from an ovarian cancer, primary colorectal cancer, appendiceal cancer, or mesothelioma. The cancer in some embodiments is in advanced stage, i.e., stage 3 or stage 4 cancer. In other embodiments, the cancer is localized. The cancer may be metastasized.

The foam compositions described herein can be administered to the peritoneal cavity using the devices described herein that have been adapted for delivery of the foam composition to the peritoneal cavity. The foam can be administered directly to the area during surgery or subsequently through a catheter placed either during or after surgery. The catheter can be an indwelling peritoneal catheter or other type of catheter. The foam composition can be administered to the peritoneal space once or multiple times.

Methods for Treating Carcinomatosis

In certain aspects, this disclosure provides methods of treating carcinomatosis by administering a foam composition to the peritoneal space of a patient in need thereof. In embodiments, the foam composition comprises one or more of mitomycin C, paclitaxel, oxaliplatin, taxotere, and cisplatin.

Carcinomatosis is a particular condition in which a neoplastic growth, malignancy, or cancer is spread throughout the body, or, in some cases, to a relatively large region of the body. In some embodiments, the malignancy is diffusely disseminated on the mesentery of the abdominal cavity. The patient suffering from carcinomatosis may have simultaneous developments of multiple carcinomas, usually after dissemination from a primary source.

The foam compositions described herein can be administered to the tissues or organs affected by carcinomatosis using the devices described herein that have been adapted for delivery of the foam composition to such affected tissues or organs. The foam can be administered directly to the area during surgery or subsequently through a catheter placed either during or after surgery. The catheter can be an indwelling peritoneal catheter or other type of catheter and the foam composition can be administered once or multiple times.

Combination Therapies

The foam compositions disclosed herein can be administered in combination with a second line of therapy to patients in need thereof. For example, in some embodiments, the foam composition is administered to a subject along with another anti-neoplastic agent including but not limited to cytotoxic chemotherapeutic agents, targeted small molecules and biological molecules (e.g., immunostimulants, antibodies, antibody-drug conjugates). The foam compositions can also be administered before or after radiation therapy and/or surgery. In some embodiments, the foam composition is administered before, during, and/or after an anti-inflammatory agent or an immunosuppressant.

Examples of cytotoxic chemotherapeutic agents that may be administered in combination with the foaming compositions described herein include cisplatin, dacarbazine (DTIC), dactinomycin, irinotecan, mechlorethamine (nitrogen mustard), streptozocin, cyclophosphamide, carmustine (BCNU), lomustine (CCNU), doxorubicin (adriamycin), daunorubicin, procarbazine, mitomycin, oxaliplatin, cytarabine, etoposide, methotrexate, 5-fluorouracil, vinblastine, vincristine, bleomycin, paclitaxel (taxol), docetaxel (taxotere), aldesleukin, asparaginase, busulfan, carboplatin, cladribine, dacarbazine, floxuridine, fludarabine, hydroxyurea, ifosfamide, interferon alpha, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil, taxol and combinations thereof.

Targeted small molecules and biological molecules include, without limitation, inhibitors of components of signal transduction pathways, such as modulators of tyrosine kinases and inhibitors of receptor tyrosine kinases, and agents that bind to tumor—specific antigens. Non-limiting examples of growth factor receptors involved in tumorigenesis are the receptors for platelet-derived growth factor (PDGFR), insulin-like growth factor (IGFR), nerve growth factor (NGFR), and fibroblast growth factor (FGFR), and receptors of the epidermal growth factor receptor family, including EGFR (erbB1), HER2 (erbB2), erbB3, and erbB4. Examples of antibodies that can be administered in combination with the foaming composition may include CD20 antibody, PD-1 antibody, and/or PD-L1 antibody.

Kits

Kits of present invention can include any combination of agents, compositions, components, reagents, administration devices, or mechanisms, or other entities provided herein. For instance, a kit of the present invention may include one or more foam composition, an administration device, and a combination therapy agent. Kits may further include a device to facilitate delivery. Any of the kits provided herein can be included in a container, pack, or dispenser together with instructions for administration.

### EXAMPLES

Hyperthermic intraperitoneal chemotherapy (HIPEC) has achieved significant survival benefits compared to other treatment modalities in peritoneal carcinomatosis patients. The mechanisms of action of this therapy are not well defined and the contributions of tumor debulking, intraperitoneal chemotherapy and heat are unknown. HIPEC is typically performed in the operating room in combination with an abdominal cancer operation. HIPEC requires significant operative technical resources and can result in increased operative time, morbidity, and cost. An example of a foam delivery system disclosed herein was administered as a treatment for peritoneal carcinomatosis in comparison to HIPEC using a well-established murine model for carcinomatosis peritonei.

Tumor model employed was BALB/c mice inoculated with CT26-Luc cell line via intraperitoneal (IP) injection.

Five experimental conditions were tested in mice having tumor inoculation and subsequently treated via IP: (i) Control (no manipulation following CT26-Luc IP injection); (ii) Sham L23 (L23 foam without chemotherapeutic); (iii) MIVIC HIPEC (HIPEC with 8 μg mitomycin C); (iv) MMC (8 μg mitomycin C); and (v) MMC L23 (L23 foam formulation with 8 μg mitomycin C). All groups were treated with the designated therapy once for the entire experiment period.

On Day 0, CT26-Luc cells were inoculated through IP injection into BALB/c mice. HIPEC procedure, sham and drug treatments were administered on Day 2. The L23 foam compositions were delivered using a pipette (total 2 mL) through a small incision as HIPEC surgery. The L23 foam compositions comprise Propylene Glycol (15%); Lipocol L-23 (0.5%); Emulsifying Wax, NF (0.5%); Cetyl Alcohol (0.3%); Methylparaben (0.05%); Propylparaben (0.01%); Propylene Glycol (5%); and Xanthan Gum (0.3%). The abdominal wall of mice that were treated with foam was closed without shaking.

From Day 3-30 in vivo luciferin imaging was performed twice a week. Upon the development of metastatic tumors, tumor presence was analyzed by in vivo imaging for luciferase activity in the left kidney and liver. Mice were anesthetized and injected IP with 100 ml of luciferin substrate 30 mg/ml solution and Luciferase activity detected 5 min after substrate injection. Luciferase activity was analyzed on IVIS Lumina-III pre-clinical in vivo imaging system.

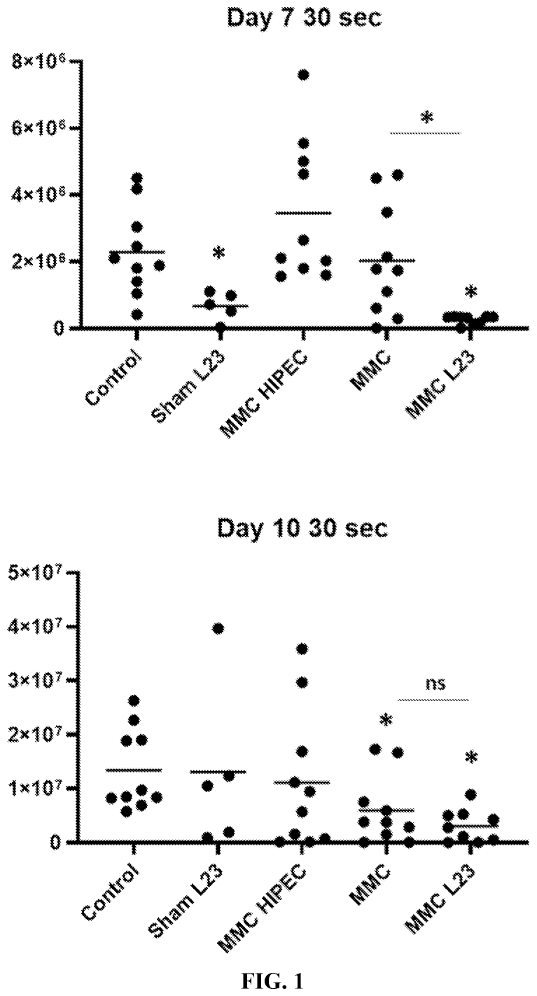

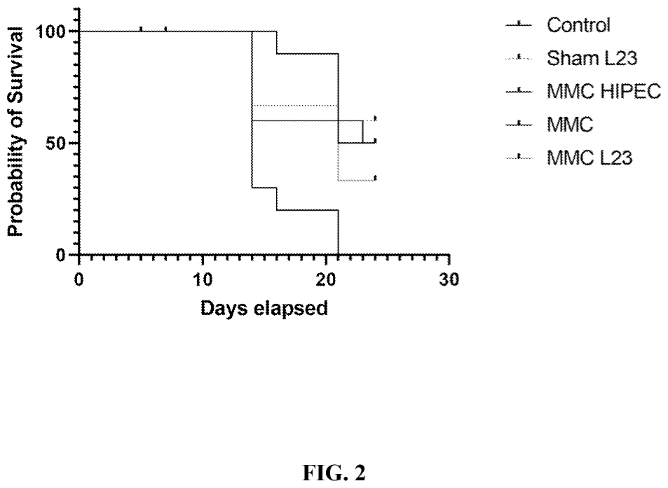

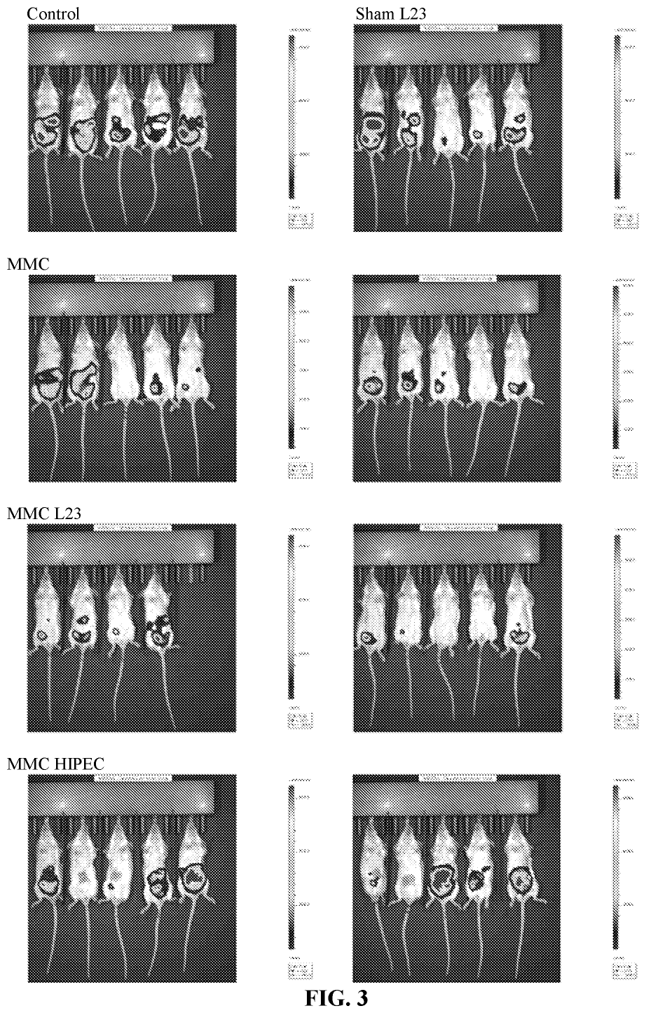

These results suggest that the foam with the chemotherapeutic agent (mitomycin C) provides a significant reduction in tumor activity compared with MMC or HIPEC with MIVIC on Day 7 (FIG. 1, top panel). On Day 10 the foam MMC composition provided a significant reduction in tumor activity compared with HIPEC with MIVIC and a reduction in tumor activity compared to MMC that did reach statistical significance at this sample size (FIG. 1, bottom panel). The foam MIVIC composition provided increased survival versus the control, as did the sham and other treatment groups (FIG. 2). Representative images are provided in FIG. 3. These results indicate that the foam delivery systems described herein for administering anti-cancer therapeutic agents to a body cavity, organ, or tissue of a patient are a promising alternative to conventional therapy.

We claim:

1. A method to treat a neoplastic disease in a patient in need thereof by administering a foam composition comprising a liquid component, a foaming agent, and one or more therapeutic agents.

2. A method of claim 1, wherein the neoplastic disease is cancer.

3. The method of claim 2, where the cancer is present in an internal organ or in a body cavity of the patient.

4. The method of claim 1, wherein the cancer is present in an internal organ.

5. The method of claim 1 or 2, wherein the cancer is bladder cancer, lung cancer or ovarian cancer.

6. The method of claim 2, wherein the cancer is present in a body cavity.

7. The method of claim 1, 5 or 6, wherein the neoplastic disease is mesothelioma or peritoneal cancer.

8. The method of any one of claims 1-7, wherein the cancer is metastasized.

9. The method of any one of claims 1-8, wherein the cancer features carcinomatosis.

10. The method of any one of claims 1-9, wherein the cancer is localized.

11. The method of any one of claims 1-9, wherein the cancer is stage 3 or stage 4.

12. A foam composition for treating a neoplastic disease comprising a liquid component, a foaming agent, a bubble-forming component, and one or more therapeutic agents.

13. The composition of claim 12, wherein the bubble-forming component is a volatile liquid or a compressed gas.

14. The composition of claim 13, wherein the gas is a single gas.

15. The composition of claim 13, wherein where the gas is a mixture of gasses.

16. The composition of claim 12, wherein the volatile liquid comprises one or more fluorinated hydrocarbons.

17. The composition of claim 16, wherein where the one or more fluorinated hydrocarbons is selected from one or more of a trifluoromethane, difluoromethane, difluoroethane, tetrafluoroethane, heptafluoroethane, perfluorobutane, perfluorocyclobutane, perfluoropentane, perfluorohexane, perfluoroheptane, perfluorooctane, perfluorocyclopentane, perfluorocyclohexane, heaxafluoropropane, and heptafluoropropane.

18. The composition of any one of the claims 12-17 where the volatile gas is a haloalkane.

19. The composition of claim 12, wherein the volatile liquid comprises H134A.

20. The composition of claim 12, wherein the volatile liquid comprises ze1234.

21. The composition of any one of claims 12-20, wherein the therapeutic agent is an anticancer agent.

22. The composition of any one of claims 12-20, wherein the therapeutic agent is selected from chemotherapeutic agents, anti-infective agents, imaging agents, hemostatic agents, gene therapy vector, hemolytic agents, adhesion reducing agents, adhesives, anti-inflammatory agents, and joint lubricants.

23. The composition of any one of claims 12-20, wherein the therapeutic agent is a chemotherapeutic agent.

24. The composition of any one of claims 12-20, wherein the one or more therapeutic agents is selected from one or more of BCG, valrubicin, doxorubicin, paclitaxel, mitomycin C, gemcitabine, vinorelbine, taxotere, carboplatin, cisplatin, oxaliplatin, and pemetrexed.

25. The composition of any one of claims 12-24, wherein the composition is sterile.

26. The composition of any one of claims 12-24, where the composition forms a foam after administering to the targeted area in the patient's body.

27. A device for delivering of the composition of any one of claims 12-26 comprising a reservoir for the therapeutic agent and propellant, a foam generator, regulator, actuator, and patient interface.

28. The device of claim 27, wherein the device comprises one or more containers.

29. The device of claim 27 or 28, wherein the device comprises two containers.

30. The device of any one of claims 27-29, wherein the device comprises containers comprising (i) a foamable composition with one or more therapeutic agents and (ii) a pressurized gas canister.

31. The device of any one of claims 27-29, wherein the device comprises containers comprising (i) a foamable composition and (ii) one or more therapeutic agents and pressurized gas.

32. The device of any one of claims 27-31, wherein the compositions of the containers are combined prior to delivery to the patient.

33. The device of any one of the claims 27-33, wherein the compositions of the containers are combined during administration to the patient.

34. The device of claim 27, wherein the device comprises a single container containing a foaming agent, one or more therapeutic agents, and one or more dissolved gasses.

35. The device of claim 27, wherein the devices comprises a single container containing a foaming agent, one or more therapeutic agents and propellant.

36. The device of any one of claims 27-35, wherein the device is a single-use device.
